(19) United States
(12) Patent Application Publication
(10) Pub. No.: US 2003/0194716 A1
(43) Pub. Date: Oct. 16, 2003

(54) Device and method for performing syntheses, analylses or transport processes

(76) Inventor: Meinhard Knoll, Steinfurt (DE)

Correspondence Address: LEYDIG VOIT & MAYER, LTD, 700 THIRTEENTH ST. NW, SUITE 300, WASHINGTON, DC, 20005-3960, US

(21) Appl. No.: 10/220,894

(22) PCT Filed: Mar. 6, 2001
(86) PCT No.: PCT/EP01/02501

(30) Foreign Application Priority Data
Mar. 7, 2000 (DE) 100 11 022.3

(51) Int. Cl. C12Q 1/68; G01N 33/53; C12M 1/34

(52) U.S. Cl. 435/6; 435/7.1; 435/287.2

(57) ABSTRACT

The present invention relates to a device and a method for performing syntheses, analyses or transport processes with a process fluid. Devices and methods of this kind are used in the field of combinatory chemistry, in-situ synthesis, parallel synthesis, solid phase synthesis or the production of arrays, especially in the field of DNA synthesis, DNA analysis, for example as DNA chips, and in the field of peptide chemistry, pharmaceutical active substance screening, high throughput screening (HTS), pharmacogenomics and the like.

The present invention relates to a device and a method for performing syntheses, analyses or transport processes with a process fluid. Devices and methods of this kind are used in the field of combinatory chemistry, in-situ synthesis, parallel synthesis, solid phase synthesis or the production of arrays, especially in the field of DNA synthesis, DNA analysis for example as DNA chips and in the field of peptide chemistry, pharmaceutical active substance screening, high throughput screening (HTS), pharmacogenomics and the like. According to prior art, for example DNA arrays are produced by combinatory synthesis (in rows and columns) on a solid body. U.S. Pat. No. 5,700,637 discloses the production of cells for this purpose in a supporting material and coupling of nucleotides to this supporting material. To produce the variety of necessary oligonucleotides, a lithographic method is used for example in which more, than 400 different oligonucleotides are attached per cm² (U.S. Pat. No. 5,744,305) or more than 1000 different oligonucleotides per cm² (U.S. Pat. No. 5,445,934).

Furthermore, in prior art is known spot synthesis for producing arrays with oligonucleotides in which reagents for the synthesis are pipetted onto defined positions of a support. The washing and unblocking steps are performed by dipping the support into appropriate solutions. This is disclosed for example for sheets of cellulose paper as the support in Beck-Sickinger, G. et al “Kombinatorische Methoden in Chemie und Biologie” [=“Combinatory methods in chemistry and biology”], Spektrum Akademischer Verlag, Heidelberg, 1999, page 53.

Furthermore, the production of arrays with oligonucleotides with the aid of a moveable block having slots or channels for the supply of reagents is known from publications U.S. Pat. No. 5,561,646 and U.S. Pat. No. 5,885,837.

This prior art has some serious disadvantages however. In in-situ synthesis it is necessary, for many individual syntheses, to pipette the reagents into cells, and the outlay for the production of an array therefore becomes very great. In the case of lithographic methods, which are also very extravagant and expensive, compatibility problems arise furthermore between the reagents for the synthesis and the photo-resist materials used for the lithography. Furthermore, after synthesis the array has to be separately fitted for measurement purposes into an appropriate flow-through cell, and this increases the outlay in the production of the analysis array.

In the case of spot synthesis, in which small droplets for the synthesis are applied to a substrate, evaporation problems arise. Since the washing and unblocking steps take place by flooding the entire substrate with appropriate substances, a repeated transfer of the substrate between the device for applying the droplets and the various baths is necessary, and for this reason repeated adjustment of the substrate becomes necessary. Here, too, after the synthesis, the array has to be fitted separately into a flow-through cell.

In the case of synthesis with a moveable block which has channels for the supply of reagents, sealing problems and problems of adjustment between the block and substrate occur. Here, too, a subsequent step of fitting the array into the flow-through cell is again necessary.

The object of the invention, therefore, is to make available a device and a method for performing syntheses, analyses, or transport processes, in which the different chemical and biochemical processes can be carried out in a single flow-through device simply and in an automatable manner. This device and these methods should make possible simple handling and be cost-effective.

This object is accomplished by the device according to claim 1 and the method according to claim 41. Advantageous developments of the device according to the invention and of the method according to the invention are given in the respective dependent claims.

The present invention makes available a microfluidic system which has a planar reaction chamber which is filled actively or passively from outside with a process fluid (reagent or sample) via at least one connection. The process fluid can flow away via a further connection. This fluid system has a control interface with which a control fluid (e.g. gas) is brought into the reaction chamber. There a control fluid domain is produced which completely or partially displaces the process fluid in the reaction chamber and thus defines compartments where an interaction between the process fluid and a substrate for example (solid phase) is not possible or respectively is prevented.

This means that the control fluid domain addresses individual regions of an array, namely in locations other than where the control fluid domain is. The control fluid domain here remains at its prescribed location by bubble adhesion even when the process fluid is exchanged.

For example, the control fluid domain can also include a specific area with process fluid and thus when a new process fluid is introduced into the reaction chamber prevent the exchange of the enclosed process fluid already present for the newly introduced process fluid in the regions defined by the control fluid domain.

By means of the control fluid, the transport of the process fluid (reagent, sample) in the reaction chamber can also be effected on the basis of the displacement action (pumping action). With the present invention, therefore, a simple and inexpensive device and a simple and automatable method are made available, with which in the same miniaturised device both the synthesis and the analysis of substances can be performed, regulated locally. In particular, in addition to reagent and sample volumes in the ml-range, also small volumes in the nl- to μl-range can be realised. In particular no pipetting steps are necessary nor any expensive lithographic methods. Therefore the present invention makes available a universal technology in the form of a “lab on a chip”.

The control device (control interface) advantageously has at least one control aperture in a side wall of the reaction chamber, the aperture being completely or partially permeable by the control fluid, for example the aperture being closed by a gas-permeable membrane. The control fluid is then brought into defined regions of the reaction chamber with the aid of an excess pressure, and it can also be removed from the reaction chamber again by means of a negative pressure. If control apertures, to which a negative pressure is applied which leads to suction of the control fluid out of the reaction chamber, are disposed around control apertures to which excess pressure is applied, along these negative pressure regions the extent of the control fluid domain is limited to defined regions.

The device according to the invention can have a large number of control apertures of any shape, which are disposed for example in the form of an array. The selection of specific control apertures takes place then by placing a structured die onto the control interface for supplying the control fluid. The control fluid is then supplied only in the regions which are defined by the die.

Alternatively, specific control apertures can also have applied to them a blocking fluid (e.g. liquid medium, water, alcohol, THF or the like), the blocking fluid preventing any penetration of the control fluid through the control aperture. In this manner, specific control apertures for the control fluid can be kept open or blocked.

The blocking fluid can be applied to the control interface advantageously by means of micro-drop methods/inkjet methods, by means of electrospray methods via an electrically addressable screen system, via dispensing methods or also by means of printing methods such as screen printing.

If the blocking fluid is readily volatile, after the blocking fluid has evaporated, the same or a new control configuration can be realised by renewed application. This is true in particular of the application of the blocking fluid by micro-drop/inkjet or electrospray methods. If the blocking fluid is not very volatile, it can be removed by being blown away from the control interface e.g. with the aid of a gas flow. It is also possible to extract the blocking fluid with the aid of additional channels integrated into the control interface.

The control fluid can, however, also alternatively be introduced into the reaction chamber via the electrochemical generation of gas by means of electrodes inside the reaction chamber. By appropriate arrangement of electrodes with voltages applied only to selected electrodes, generation of control fluid and control fluid domains at selected locations in the reaction chamber is possible on the control interface.

The side walls of the reaction chamber are advantageously flat solid bodies which delimit the reaction chamber. They can advantageously consist of plastics material, glass, ceramics and the like and have a planar, porous or structured surface. Side walls of this type can also be used as the reaction interface, the desired reactions, for example synthesis reactions, then taking place at this surface. Alternatively the reaction interface can also be realised in the reaction chamber by means of particles, fabric, mats or other materials being applied to the side wall or being introduced into the volume of the reaction chamber.

One or both of the side walls of the reaction chamber can be configured as the analysis interface, a flat solid body again being suitable for this. This can serve for example as a support for electrochemical sensory analysis according to prior art, or also be optically transparent in order to perform optical analysis.

By this means, for example, the device according to the invention can be filled according to a previously performed selective site-specific synthesis of various molecules (subsequently called analysis molecule) with a fluid containing the target molecule and the interaction with individual synthesised analysis molecules can be examined. For this purpose are suitable all the conventional assay formats, for example with fluorescence-marked molecules or with an enzyme-marked molecule, for example with an array-like arrangement of different oligonucleotides.

The analysis interface and the reaction interface can here also be identical, and during the synthesis of the analysis molecule, for example an array of oligonucleotides, one of the side walls of the reaction chamber serves as the reaction interface, which is then used as the analysis interface to detect and analyse a target molecule.

The device according to the invention can have lateral dimensions in the range between several mm and several cm; individual array elements, which are defined by the control fluid domains, can be of the order of magnitude of 0.001 mm to several millimetres. The size of the array elements can be altered by adjusting the size of the control fluid domain. To this end, the control plate can have a thickness of several μm to several mm, the gas-permeable membrane a thickness of several 100 nm to several 100 μm, the analysis or reaction interface can have a thickness of several μm to several mm and the control apertures a diameter of several μm to several mm.

The height of the reaction chamber between the control interface and the analysis interface can be between several 10 μm and several mm.

Suitable as materials for the control device are plastics material, glass, ceramics or even a sealing layer locally applied to a gas-permeable membrane. As the gas-permeable membrane are suitable silicon, Teflon and the like; for the analysis interface glass, polycarbonate, polyvinyl chloride, polypropylene, polyurethane, polyester and the like; for the reaction interface polymers, synthetic resins, polycarbonate, glass, ceramics and the like. As the control fluid, gases such as noble gases, e.g. argon or nitrogen can be used. It is essential here that this gas is so selected that it is compatible with the process fluid (reaction fluid). Liquid materials such as water, alcohol, THF or other fluid media are suitable as the blocking fluid.

The side walls of the reaction chamber can be designed flat and comprise for example different materials with different surface tensions, which alternate where the control fluid domain boundaries occur later. This can contribute to an improved control fluid domain adhesion (bubble adhesion). They can be microstructured or designed strip- or fibre-shaped. Their surfaces can be modified, for example to immobilise chemical, biological or biological components. Furthermore as the reaction interface particles, mats or fabrics are suitable which are applied to one of the side walls of the reaction chamber or are introduced into the volume of the reaction chamber.

The control interface and the permeable membrane can be securely interconnected as part of the flow-through device. Alternatively it is also possible for the control interface to be moveable as part of the system unit and be placed on the gas-permeable membrane. The control aperture can be shaped round, square, conical or in any other way, according to the desired control fluid domain.

By means of the control fluid domains, i.e. by means of the arrangement of the control devices, for example the control apertures or the electrodes, it is now possible to produce arrays with n lines and m columns of process areas which can be separated from one another by control fluid domains. Thus, for example, arrays with 100×100 or 10000×10000 process areas are possible.

The device according to the invention can be produced by means of injection moulding, micro-stamping, LIGA methods, by means of film-lamination or by connecting the individual layers of the side walls, control interface, analysis interface and process interface, the gas-permeable membrane, the channel support and the like by means of gluing, lamination, or also as one piece.

The device according to the invention and the method according to the invention consequently permit the handling of fluids and the performance of chemical and biochemical reactions for syntheses and analyses in a single flow-through device. This flow-through device can be operated with the aid of a system unit which supplies for example the process fluids, control fluid, blocking fluid and the like to the device. Furthermore the device can have a temperature-control device and/or devices for analysis, e.g. light sources or detectors.

It is advantageous about the present device and the present method that a simple flow-through device is available as the microfluidic system for relatively large-surface systems with very many elements. This flow-through device can also be produced as an inexpensive single-use article and then be used in conjunction with an appropriate system unit. It is possible, as a result of the generation of any number of reaction areas, to perform in parallel in-situ syntheses or a multi-analysis in the same device. In particular, synthesis and analysis are possible in the same device, either in parallel or one after the other chronically, which makes redundant the conversion of an appropriate synthesis device to an appropriate analysis device.

Because the gas bubbles of the control fluid can be generated in any size, a high degree of integration and thus the realisation of large arrays is possible with small surface elements.

Some embodiments of the present invention are described below.

The figures show

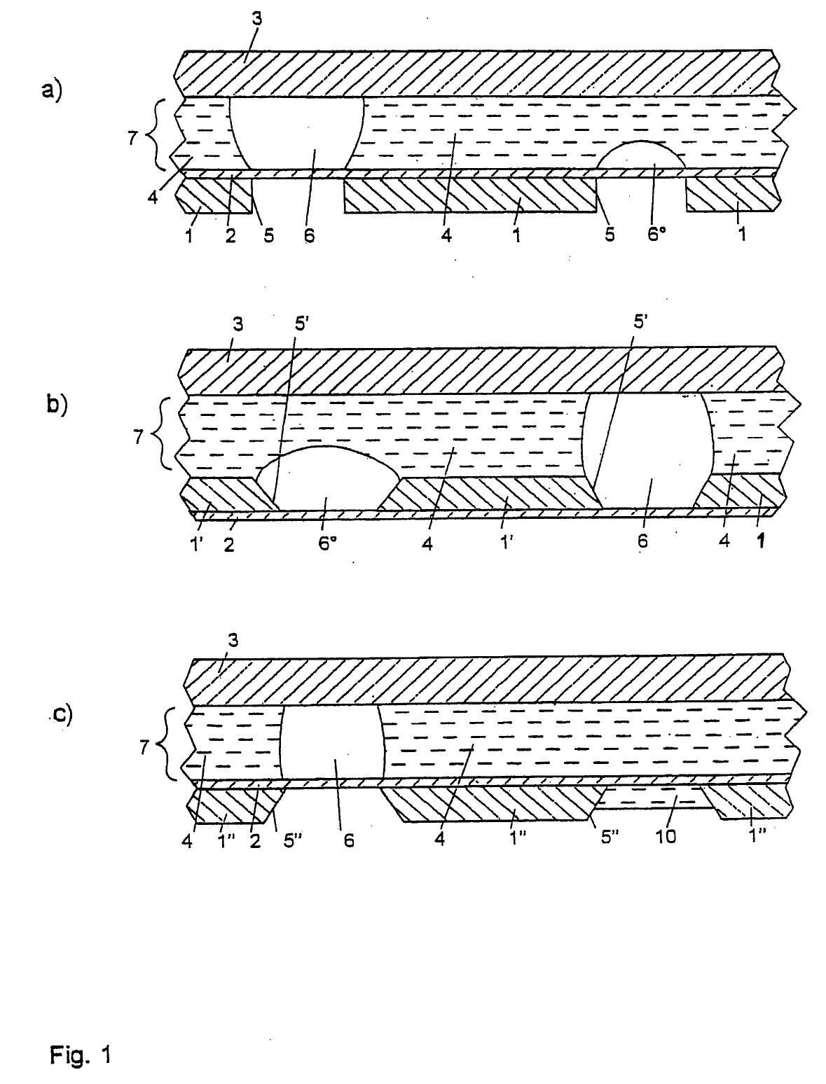

FIGS. 1 to 11 different embodiments of the device according to the invention;

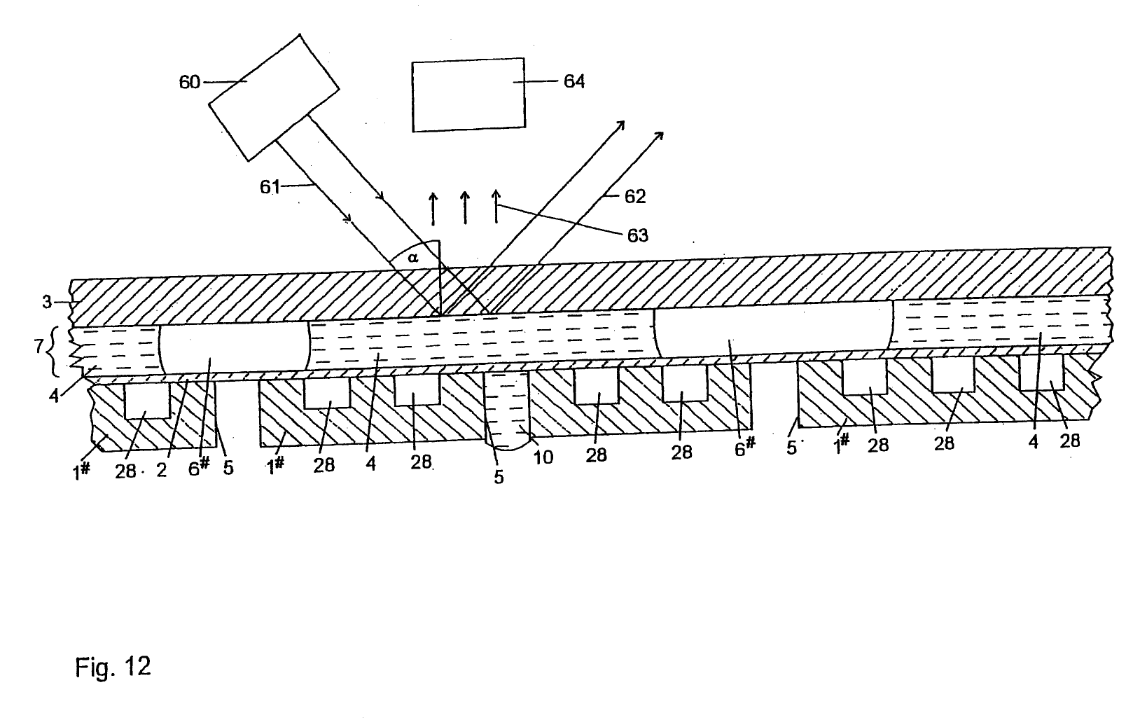

FIG. 12 an optical analysis method;

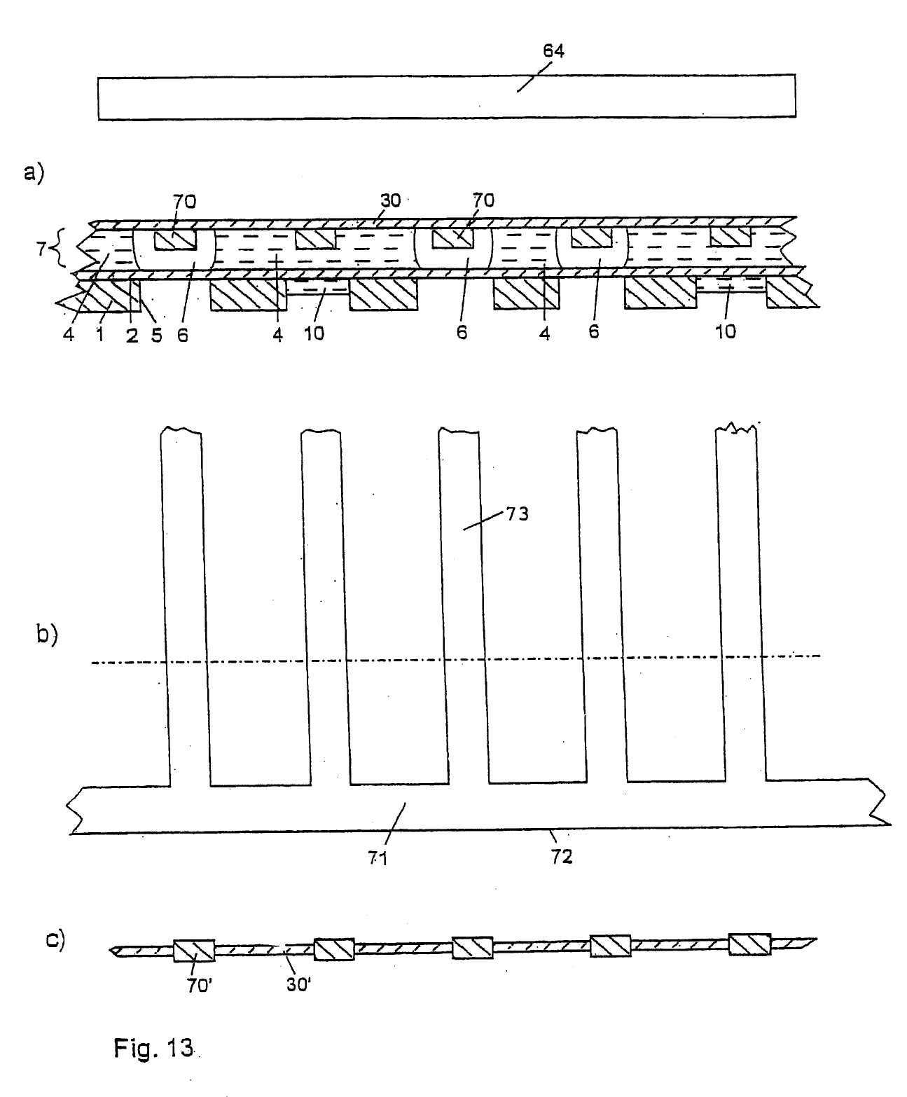

FIG. 13 a substrate in the form of a light guide;

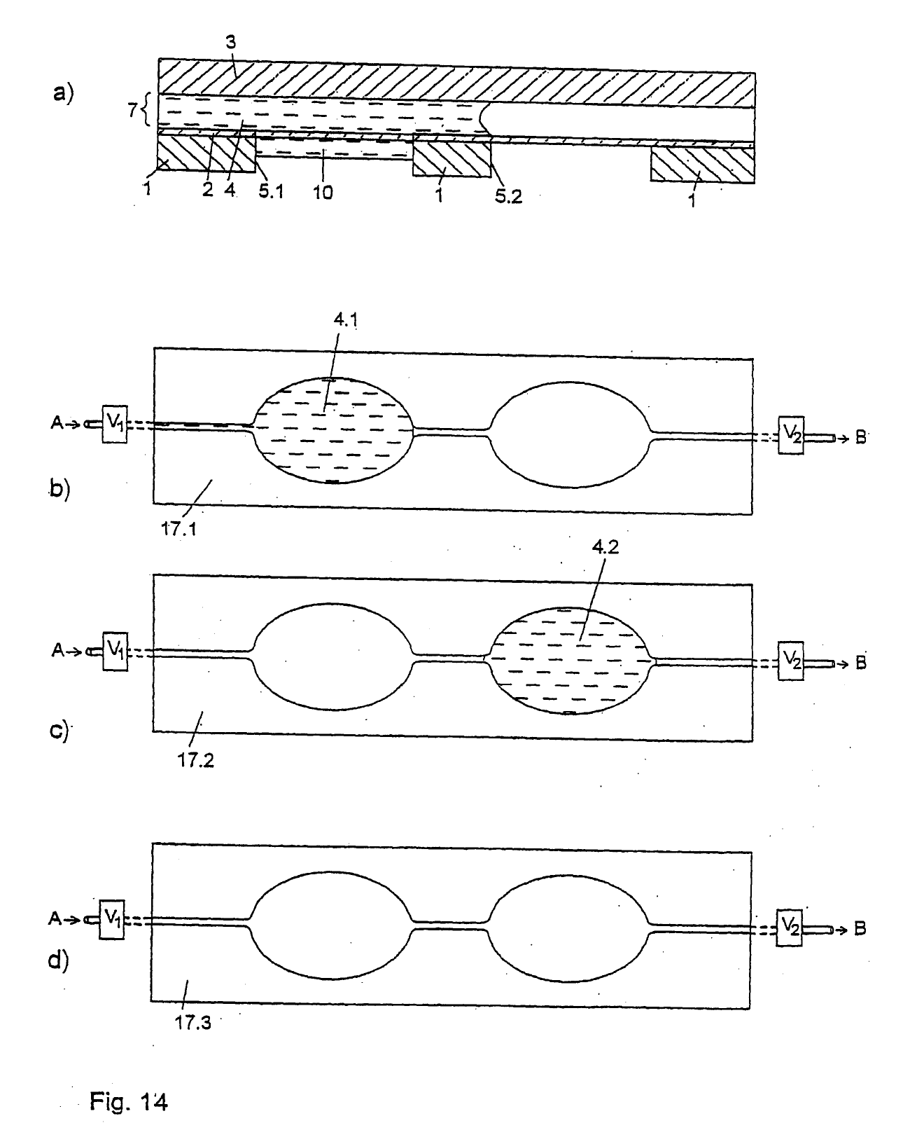

FIG. 14 a pumping method;

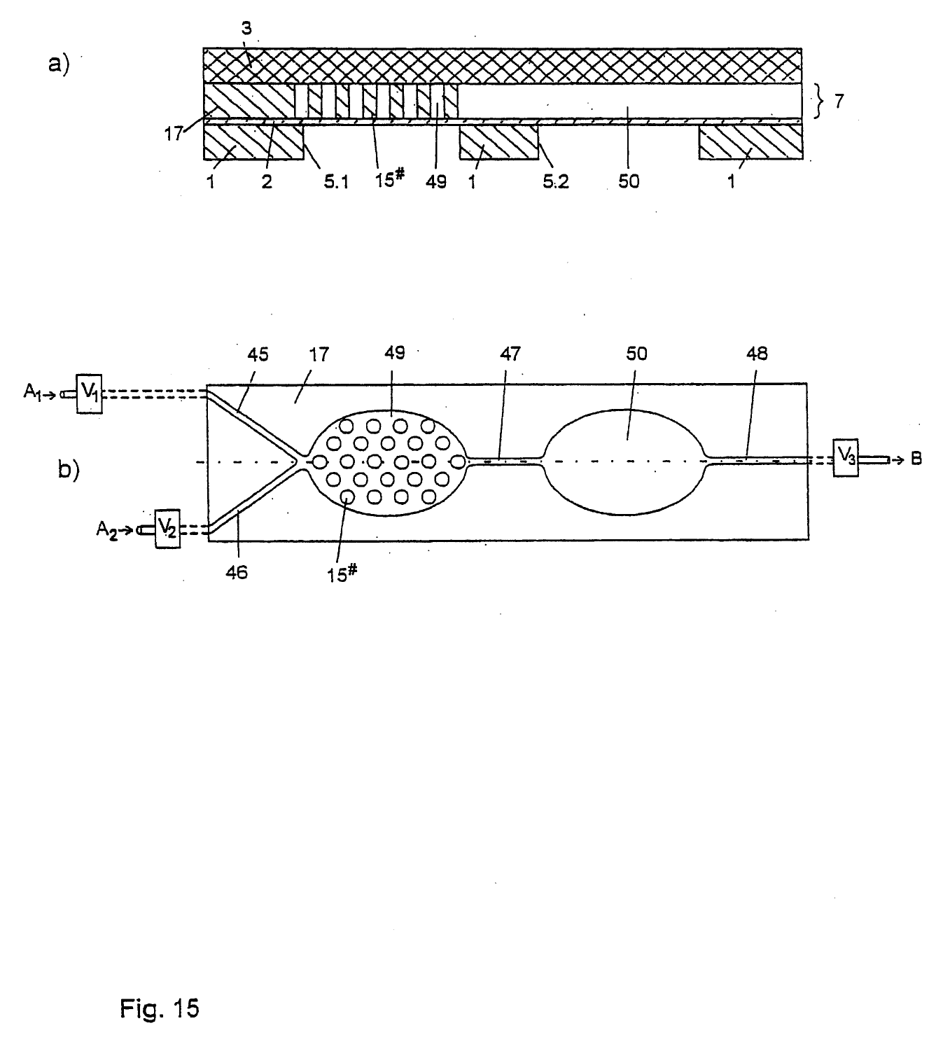

FIG. 15 a pumping and mixing process;

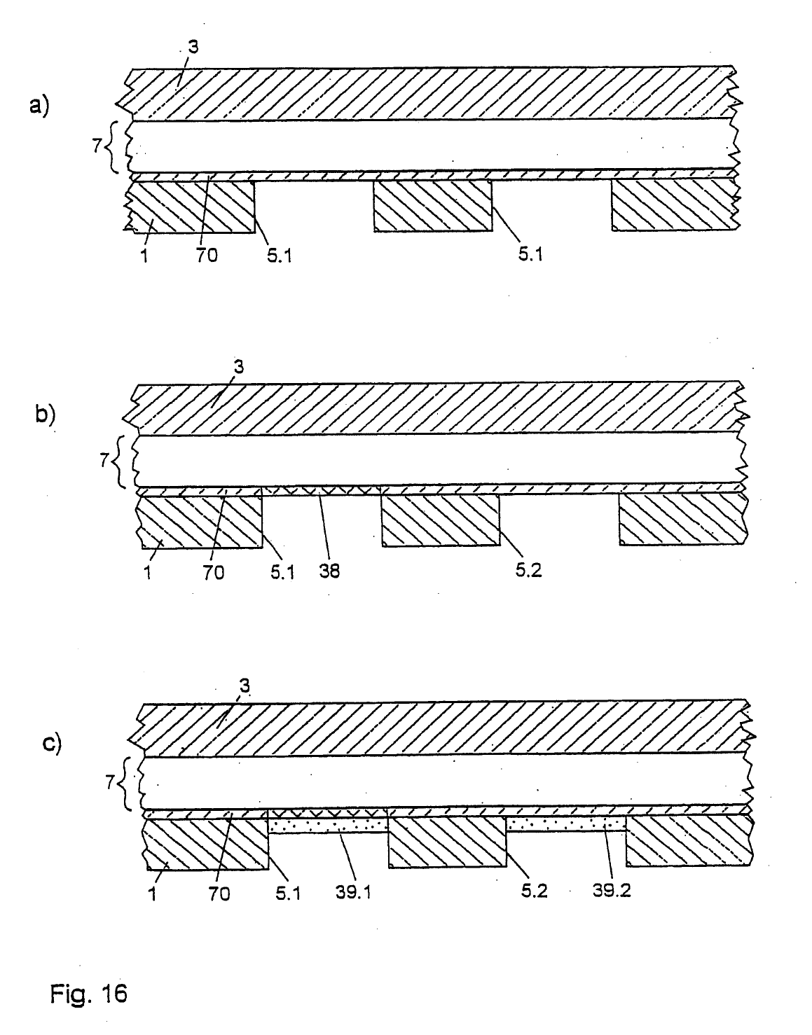

FIG. 16 the introduction of functional layers at selected locations; and

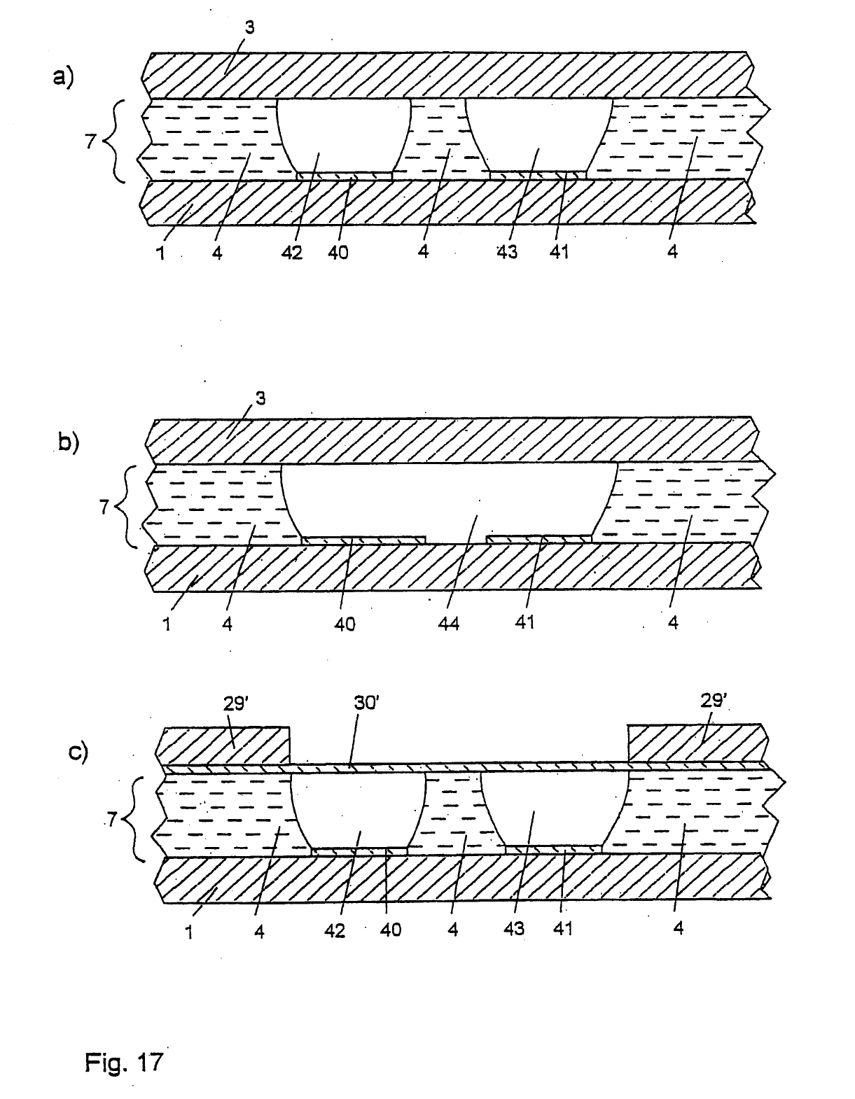

FIG. 17 the electrolytic production of control fluid.

In FIG. 1a is represented a planar reaction chamber 7 which is delimited by two side walls 3 or respectively 1 and 2. The one side wall is here formed from a control plate 1 on which a gas-permeable membrane 2 is disposed on the side facing the reaction chamber 7. In the control plate 1 are inserted control apertures 5, through which the gas-permeable membrane 2 lies open to the outside. The other side wall of the reaction chamber 7 is formed by a plate-shaped analysis interface 3.

In the reaction chamber 7 is located a process fluid 4, which is only interrupted by gas bubbles 6 of a control fluid (control fluid domains 6, 6°).

According to the invention, the device is so operated that, with the aid of excess pressure, a control fluid can be brought into the reaction chamber through the control aperture 5 and the gas-permeable membrane 2. This control fluid then forms a control fluid domain 6 in the reaction chamber, which displaces the process fluid 4 from these sub-areas. Consequently no reaction of the process fluid 4 can take place there. This is also true during an exchange of the process fluid since the control fluid domains remain stationary as a result of the bubble adhesion.

In FIG. 1a are represented two different control fluid domains 6, 6°, control fluid domain 6 forming a double-contact domain with contact both to the control interface 1 and to the analysis interface 3. In contrast, the contact control fluid domain 6° only has one contact to the control interface 1, 5. The different control fluid domain shapes serve here only as an illustration. Naturally similar control fluid domains occur if the process is carried out with, the same control apertures at the same excess pressure in the region of the control apertures.

The contact between process fluid 4 and the analysis interface 3 or respectively the control interface 1, 2 gin the region of the control fluid domain 6 is prevented by control fluid domain 6. Therefore, due to the influence of the control fluid, the interaction between process fluid 4 and for example a substrate applied to the analysis interface only takes place in the regions defined by the boundaries of the control fluid domains. Even when the process fluid is changed, the control fluid domains 6, 6° remain in place due to bubble adhesion.

The analysis interface 3 can, with its inner surface, also serve, for example, as a substrate for a solid phase synthesis as described further below.

In FIG. 1b is represented a further device, however the control interface formed from control plate 1 and gas-permeable membrane 2 is constructed inversely by comparison with FIG. 1a. The control apertures 5′, widening conically in the direction of the reaction chamber, as represented in FIG. 1b, here further the adhesion of the control fluid domains 6, 6° in the region of control apertures 5′ via bubble adhesion.

Here, as in what follows, corresponding elements are described with corresponding reference numerals, so that their description is partially omitted.

FIG. 1c shows a structure which corresponds to the device of FIG. 1a; however the control aperture 5″ narrows conically in the direction of the reaction chamber 7.

A blocking fluid 10 is introduced into one of the two control apertures 5″. If an excess pressure of the control fluid is now applied to apertures 5″, the blocking fluid 10 prevents the formation of a control fluid domain in the region of the blocked aperture 5″. The blocking fluid 10 can here be applied either by placing on a die to supply the blocking fluid, by micro-drop/inkjet methods, by electrospray methods by means of an electrically addressable screen system, by dispensing methods or printing methods such as screen printing for example, to quite specific control apertures 5″.

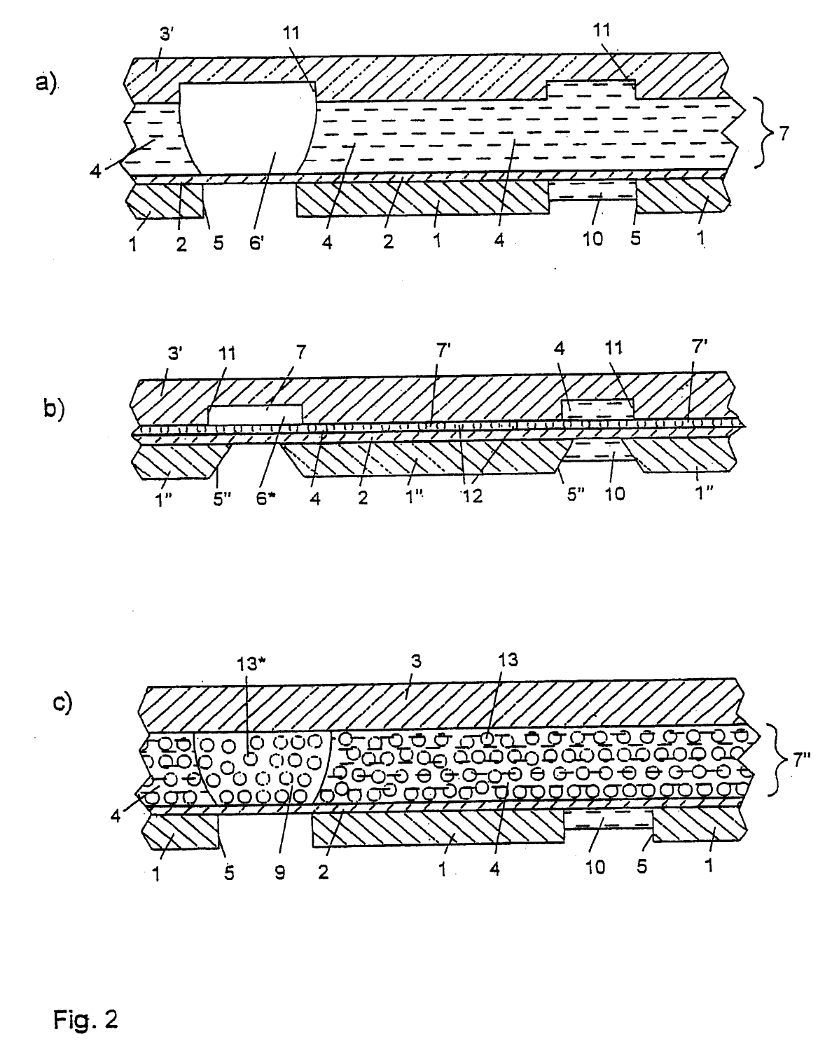

FIG. 2a shows a structure which largely corresponds to the structure of FIG. 1a. However the analysis interface 3′ has depressions 11, which lead to an improved adhesion of control fluid domains 6′ on the inner surface of the analysis interface 3′. In FIG. 2a, also, one of the control apertures 5 is blocked by means of blocking fluid, such that the formation of a control fluid domain is prevented there. In the further embodiment, the control interface comprising control plate 1 and gas-permeable membrane 2 can also be replaced by a control interface as per FIG. 1b. In this case, the height of the reaction chamber is reduced and improved adhesion of the control fluid domain on the control interface 1, 2 and also on the analysis interface 3′ is achieved.

FIG. 2b shows a structure which is analogous to the structure in FIG. 2a, but the control apertures 5″ taper conically in the direction of the reaction chamber 7′. Depressions 11 in the analysis interface 3′ now form additional reaction chambers 7. Furthermore, the reaction chamber between the gas-permeable membrane 2 and the analysis interface 3′ is covered by a matrix 12, which can consist for example of fabrics, particles, mats and the like. The matrix 12 can also serve as the substrate for a solid phase synthesis. Here, too, the formation of a control fluid domain in one of the two drawn-in control apertures 5″ is again prevented via a blocking fluid 10.

FIG. 2c shows furthermore a structure which corresponds to the one in FIG. 1a. The reaction chamber 7″ is, however, filled with a matrix 13 formed from particles, porous material, fabric layers, mat layers or the like. Here, too, a control fluid domain 9 is formed which is realised in region 13* of the matrix 13 and extends between the two side walls of the reaction chamber 7″. The matrix 13 can serve as the substrate for a solid phase synthesis.

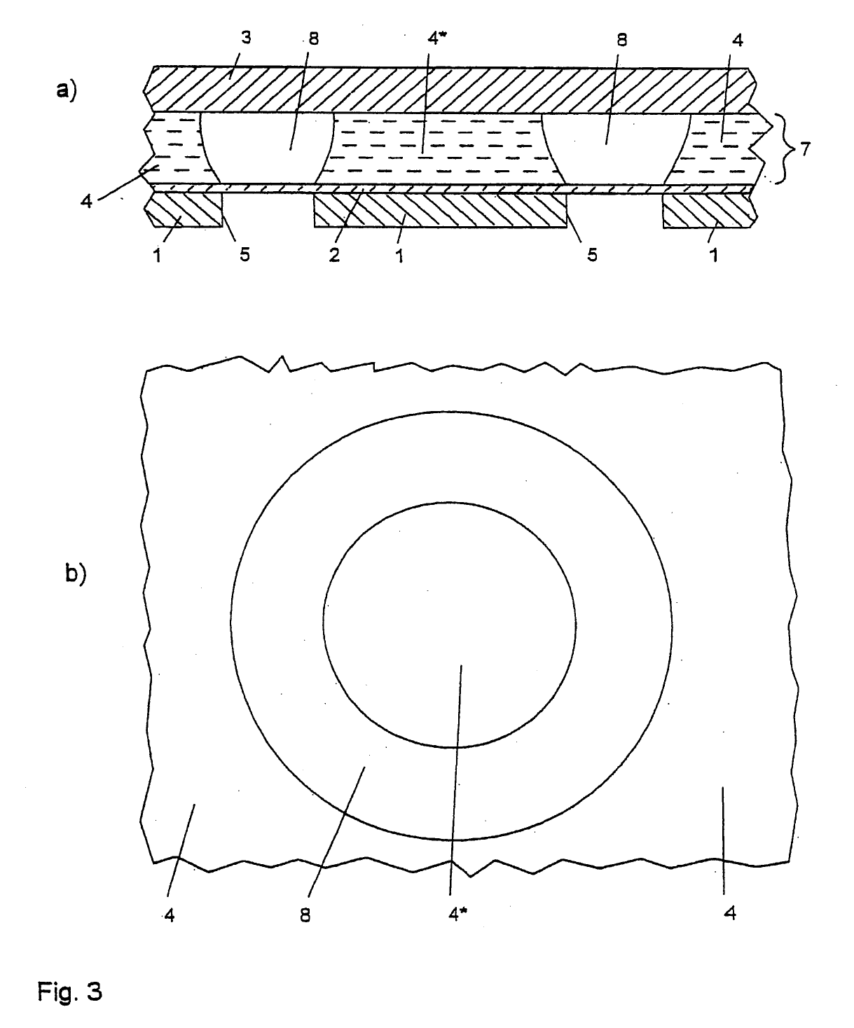

FIG. 3 shows a device firstly in side view as in FIG. 1a and also in a plan view in FIG. 3b. It can be recognised that the control apertures 5 form a ring, via which a control fluid domain 8 can be produced in the process fluid 4. Through this annular control fluid domain 8, a portion 4* of the process fluid is separated from the remaining process fluid and for example will remain there when the process fluid 4 in the outer space around the annular control fluid domain 8 is removed or exchanged.

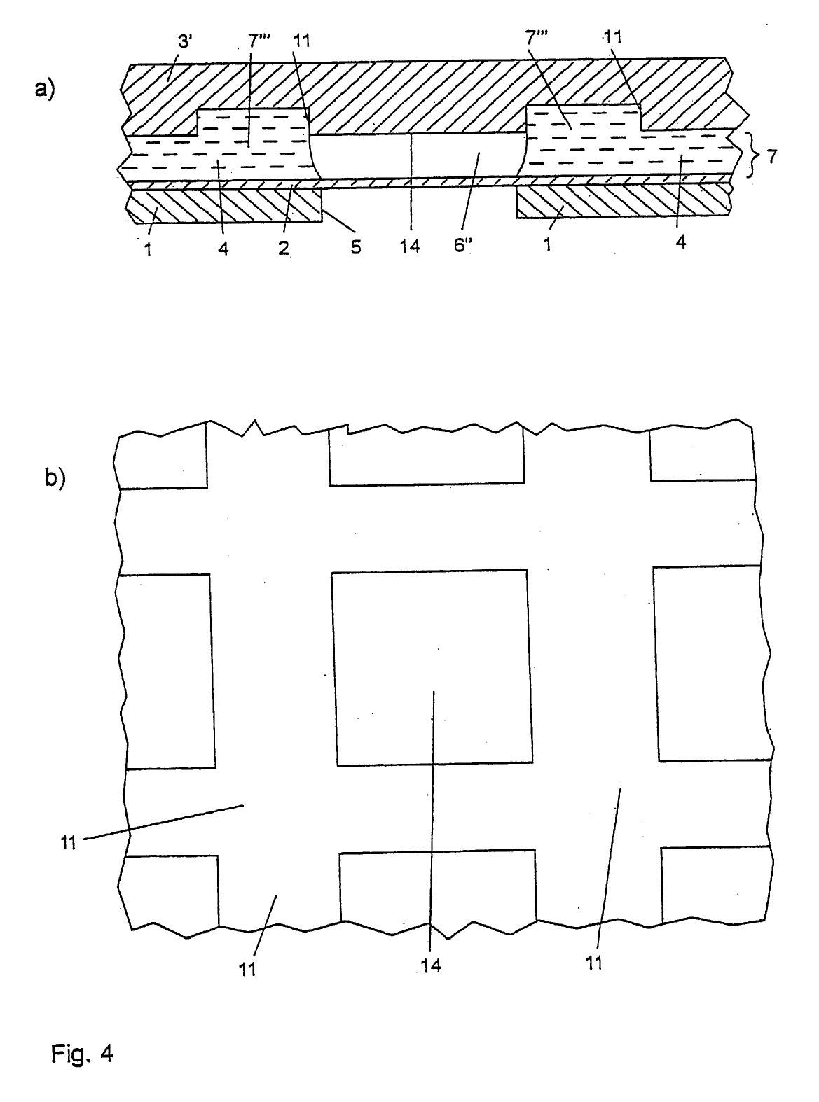

FIG. 4 shows a corresponding device to that of. FIG. 2a. The reaction chamber is now formed by the partial reaction chambers 7 and 7′″. FIG. 4b here shows a plan view of this device according to FIG. 4a, it being recognizable that the depressions 11 which form the reaction chamber 7′″, are arranged in the form of a cross-line pattern. Thus in the region of the inner surfaces 14 which are opposite the control apertures 5 on the side of the analysis interface 3′, rectangular regions can be excluded from the reaction chamber 7 with the aid of the control fluid domains.

Altogether, as a result of the arrangement of the rectangular regions 14, an array of any size is produced from individual rectangular fields 14 (array elements). In the production of oligonucleotide arrays, each array element can be taken individually, by means of control fluid domains 6″, from the respective ligation step for an additional nucleotide, such that in succession each array element can obtain a specific oligonucleotide. It is then possible to make the target substance flow over the entire array and to scan, by means of the analysis interface 3′, the entire array for corresponding specific reactions between an oligonucleotide and the target substance. Due to the arrangement according to FIG. 4, it is possible with a relatively large reaction chamber height in region 7′″ to produce a small gap between the gas-permeable membrane 2 and the analysis interface 3′, at least in region 14, in which control fluid domains should be produced.

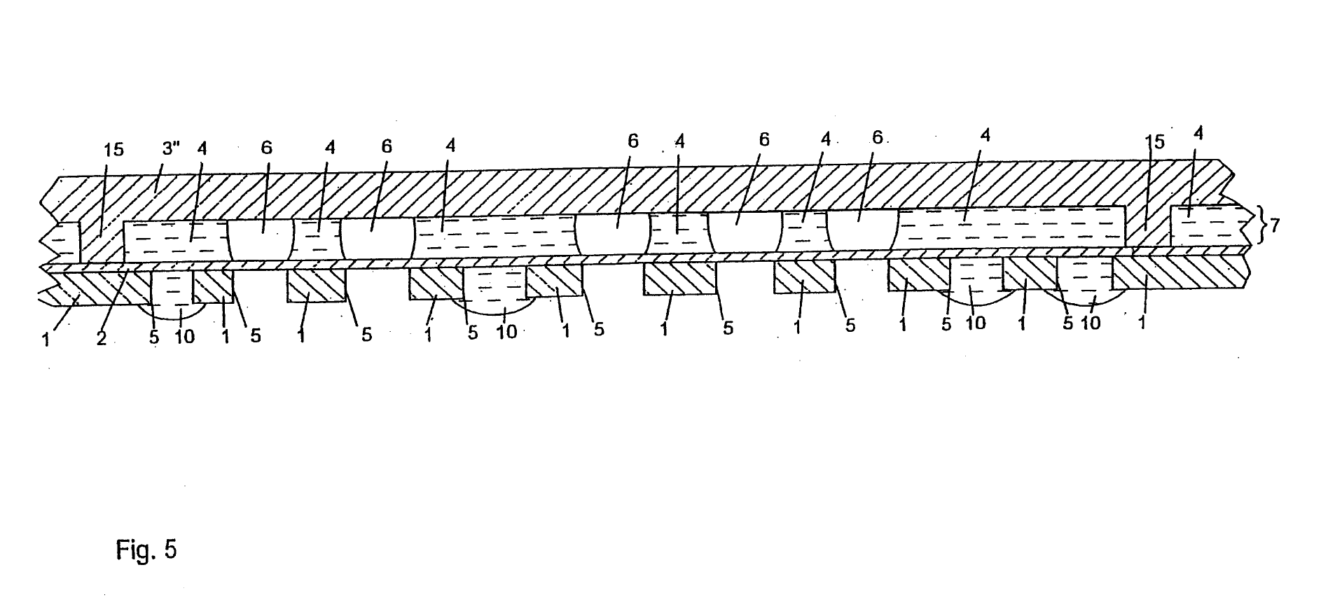

FIG. 5 shows a further device which corresponds to that one of FIG. 1a, but a larger number of control apertures 5 are provided. The spacing between the control interface 3″ and the gas-permeable membrane 2 is here guaranteed by spacers 15, which extend as webs from the analysis interface 3″ in the direction of the gas-permeable membrane. In this example, some of the control apertures 5 are occupied by blocking fluids 10, such that above these occupied control apertures 5 no control fluid domain 6 can be formed. In these regions, therefore, the process fluid 4 reacts with the analysis interface 3″ or respectively, possibly, with the gas-permeable membrane 2.

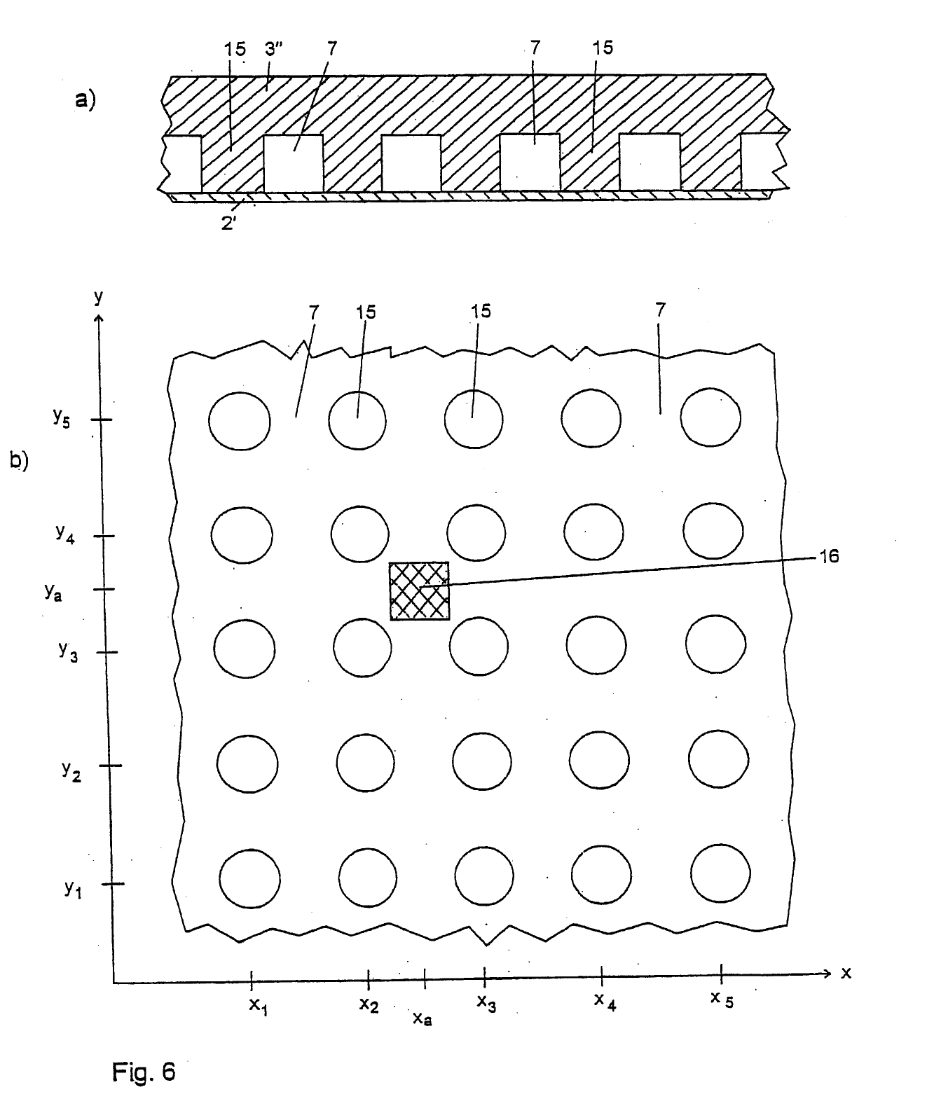

FIG. 6 shows a detail of a similar device to that of FIG. 5a. The spacing between the analysis interface 3″ and the gas-permeable membrane 2′ is guaranteed by a large number of spacers 15. The spacers 15 are here disposed in the form of a matrix. If there is one control aperture on the side of the control interface between each of the individual spacers 15, regions which are enclosed in a rectangular shape between respectively four spacers 15, i.e. surface elements 16, can be separated from the remaining region of the reaction chamber 7 via corresponding control fluid domains between the individual spacers 15. This is represented here for example for a fluid element with the coordinates Xₐ, Yₐ in FIG. 6b.

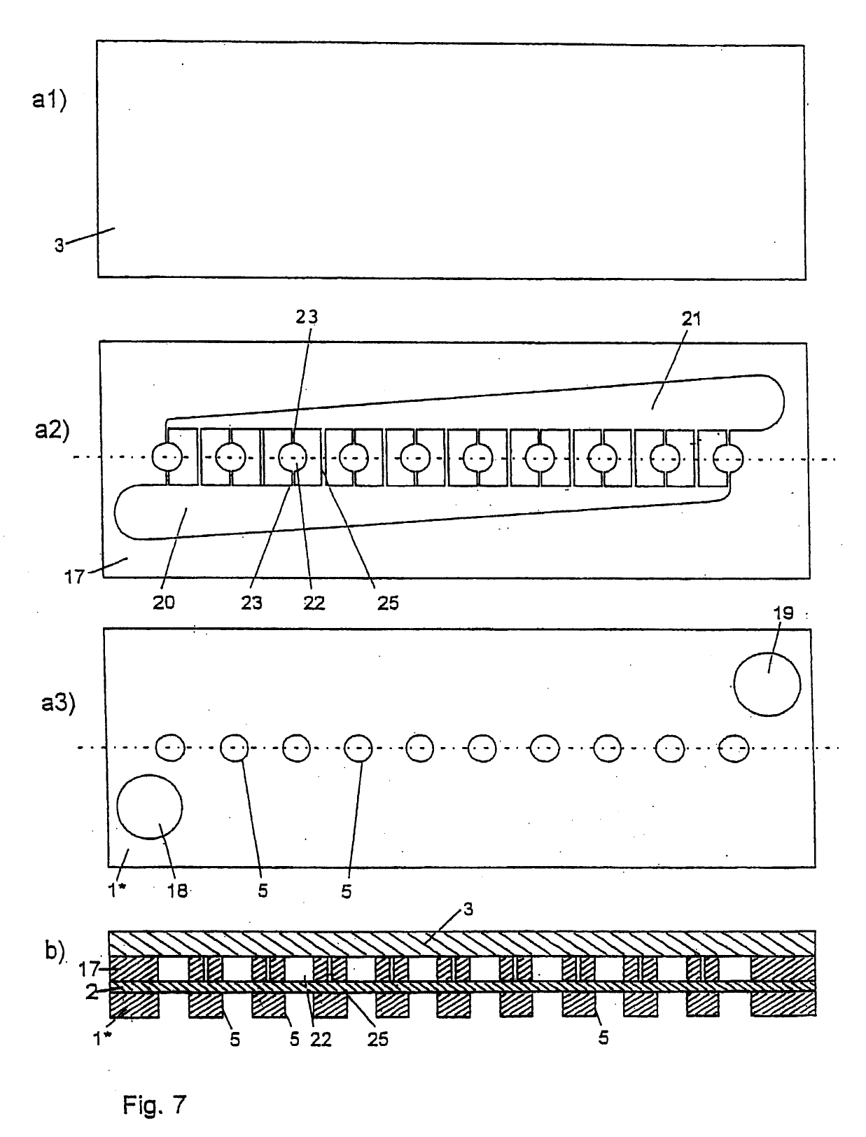

FIG. 7 shows a further device according to the invention, FIG. 7a1 representing the analysis interface 3. FIG. 7a2 shows a channel support 17 disposed between the analysis interface 3 and control interface 1#, into which a feed channel 20 and a discharge channel 21 are introduced. Between the two channels extend respectively chamber supply channels 23 and 24 which lead to chambers 22. Furthermore between the respective chamber supply channels 23 and chambers 22 there are direct connections between the feed channel 20 and the discharge channel 21 as parallel channels 25. In FIG. 7a3 is represented a control plate 1# which has a feed aperture 18 for process fluid, a discharge aperture 19 for process fluid and control apertures 5 between these apertures.

FIG. 7b now shows a lateral arrangement comprising control plate 1#, with the gas-permeable membrane 2 arranged above it, above it the channel support 17 and above it the analysis interface 3. The control apertures 5 in the control plate 1# are here so disposed that they respectively come to lie below a chamber 22. The reaction chamber is now formed by the individual chambers 22, in which, however, simultaneously control fluid domains can be formed individually via the control apertures 5. In this way it is possible to release the individual chambers 22 for a process fluid to flow through or to block them. The control apertures 5 can also, as shown in the previous examples, be blocked individually via a blocking fluid and thus be blocked when an excess pressure of control fluid is applied to all the control apertures 5, and the, formation of a control fluid domain in the associated chamber 22 can be prevented.

So that when the process fluid flows, the control fluid domains are not pressed out of the chambers 22, the pressure difference in the process fluid between the chamber entrances and exits must be limited. The parallel channels 25 can help for example with this. However it is also possible to operate without such parallel channels.

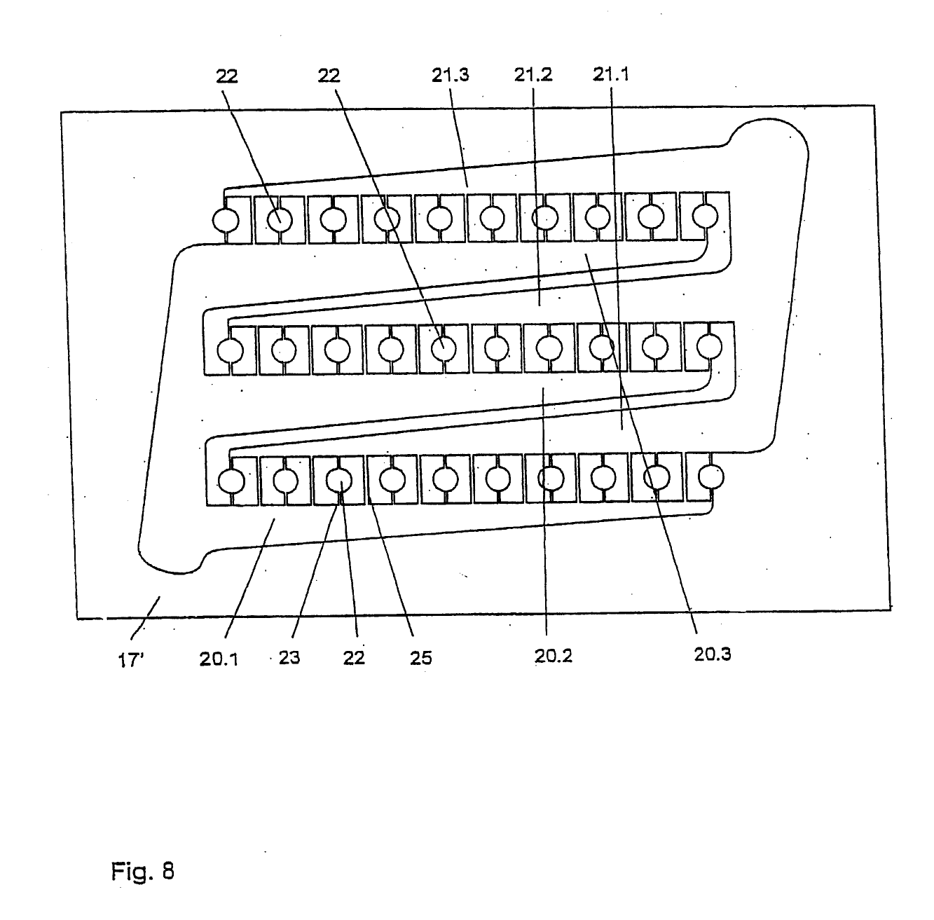

FIG. 8 shows a further device which corresponds to the device represented in FIG. 7. Here, however, a large number of rows of chambers 22 is provided. In this way the number of chambers 22 to which a process fluid is to be applied simultaneously can be further increased.

Instead of arranging one chamber 22 in each case between chamber supply channels 23 and 24, two or more interconnected chambers can be disposed in a row. Here the first chamber can, with the aid of a control fluid, assume a pseudo-valve function, which makes possible or prevents the passing of a process fluid into an adjoining chamber (e.g. reaction chamber).

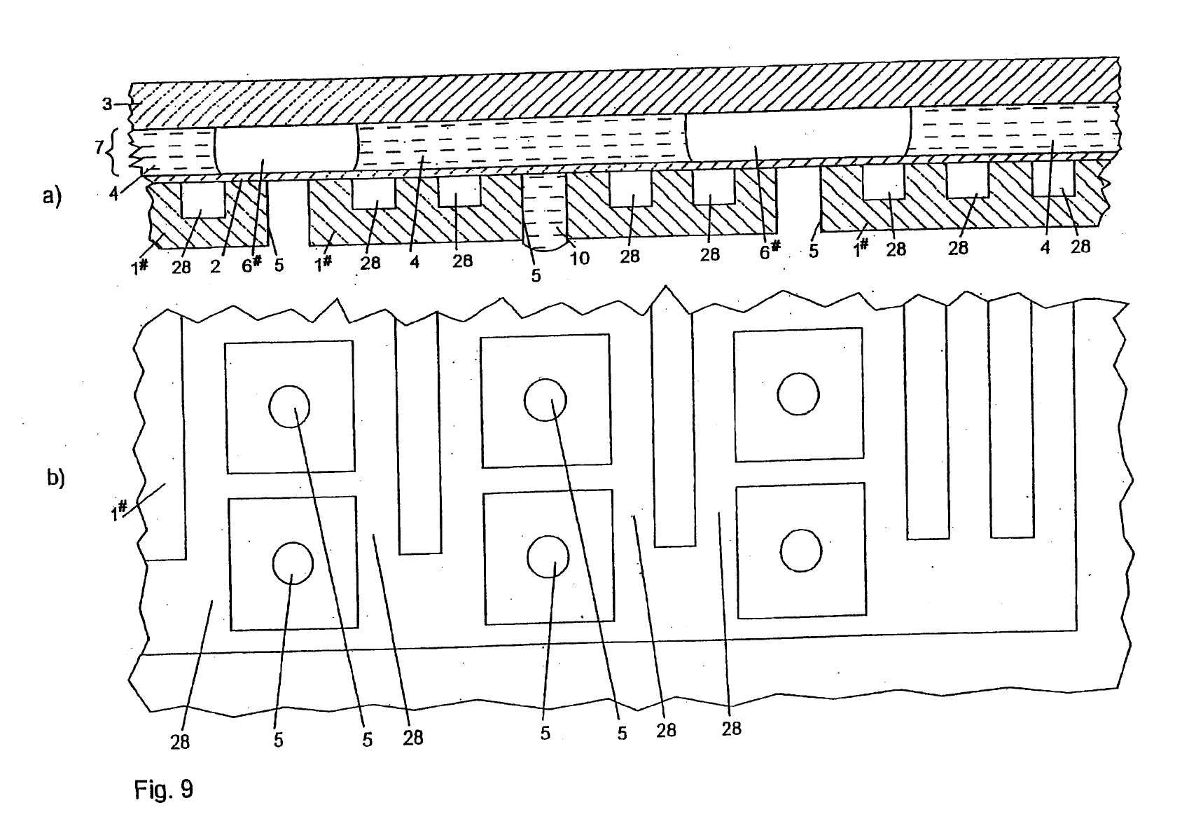

FIG. 9a is a device corresponding to the device represented in FIG. 1a. Unlike the latter however, the control plate 1# is provided with negative pressure chambers 28 which are connected to a pumping device (not shown). The negative pressure chambers 28 are here in contact with the gas-permeable membrane 2 and in each case surround laterally a control aperture 5. A plan view of this arrangement as a section through the control plate 14 is represented in FIG. 9b.

If excess pressure is applied to the control apertures 5, again above the control apertures 5 which are not blocked by a blocking fluid 10, in each case a control fluid domain 64 is formed. This control fluid domain extends also in the reaction chamber 7 at the side of the respective control aperture 5. The lateral expansion of the control fluid domain 6# is however limited by the negative pressure in the negative pressure chambers 28. For if the control fluid domain 6# moves into the region of the negative pressure chambers 28, the control fluid is extracted again from there by suction. Thus the inflow of the control fluid via control apertures 5 is in equilibrium with the outflow of the control fluid via the negative pressure chambers. Consequently a restriction of the expansion of the control fluid domains is possible via the negative pressure chambers 28.

In an analogous manner to FIG. 9, negative pressure chambers can also be introduced into arrangements according to FIG. 7 or 8. Here the negative pressure chambers are to be disposed below the chamber supply channels 23 and 24 from which they are then separated by the gas-permeable membrane.

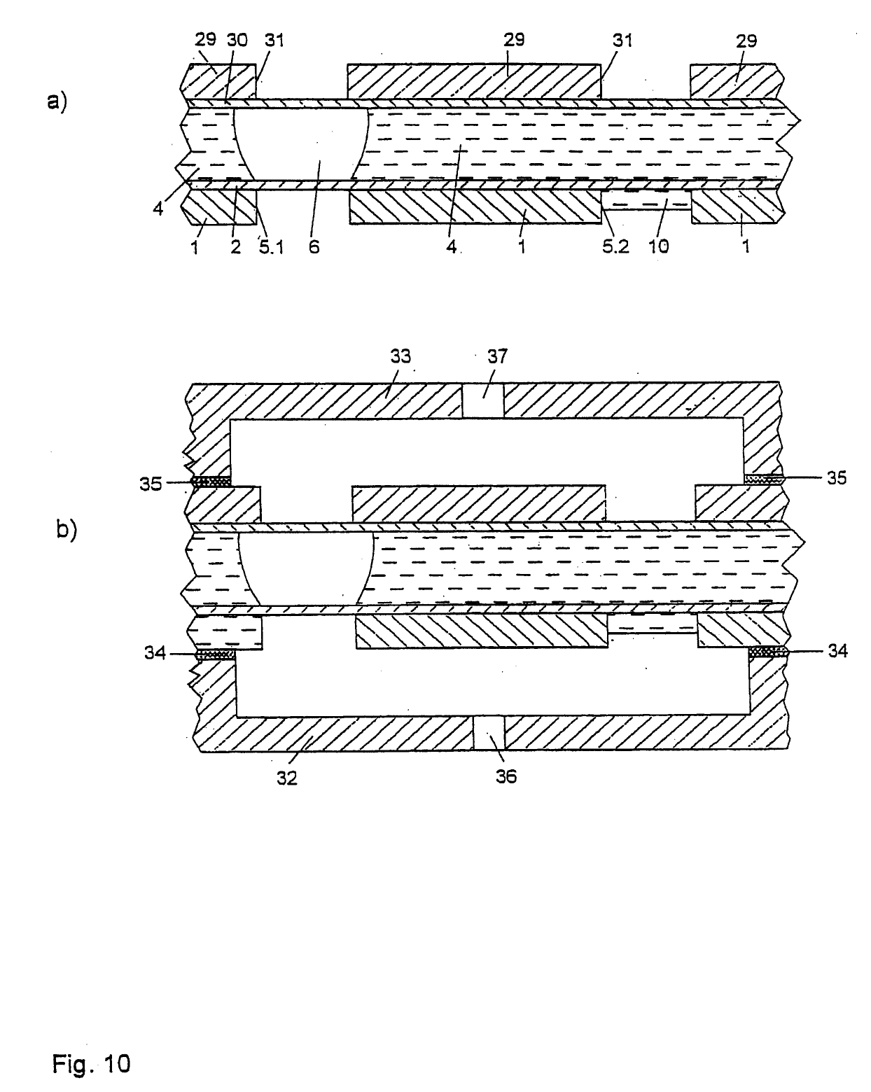

FIG. 10a shows a further arrangement which corresponds to that in FIG. 1a. Instead of the analysis interface 3, however, a second control interface is provided comprising a second control plate 29 and a second gas-permeable membrane 30 which is located on the side of the reaction chamber 7. This second control interface again has control apertures 31. According to FIG. 10a it is possible to form the control fluid domains via control aperture 5.1 or via control aperture 31. In FIG. 10a is illustrated how the control fluid domains are formed via control aperture 5.1 or respectively 5.2, control aperture 5.2 being blocked by a blocking fluid 10.

FIG. 10b shows a sectional view of this device from FIG. 10a, there being located in addition on the first control interface an extension 32 which interlocks with the first control interface, sealing by means of seal 34. On the second control interface is located an extension 33 which is disposed interlocking with the second control interface in a sealing manner via seal 35. In each of the extensions 32 or 33 is located an aperture 36 or 37.

During operation, an excess pressure of control fluid can be applied via aperture 36 to the control apertures of the first control interface, such that corresponding control fluid domains are formed in the reaction chamber. By applying a negative pressure to aperture 37, the control fluid is extracted by suction again from the control fluid domains formed, such that the expansion of the control fluid domains can be limited by regulating the negative pressure on the second control interface. The size of the control fluid domain, where control aperture 5.1 and 31 are of the same size, arises from the relationship between excess pressure in extension 32 and negative pressure in extension 33.

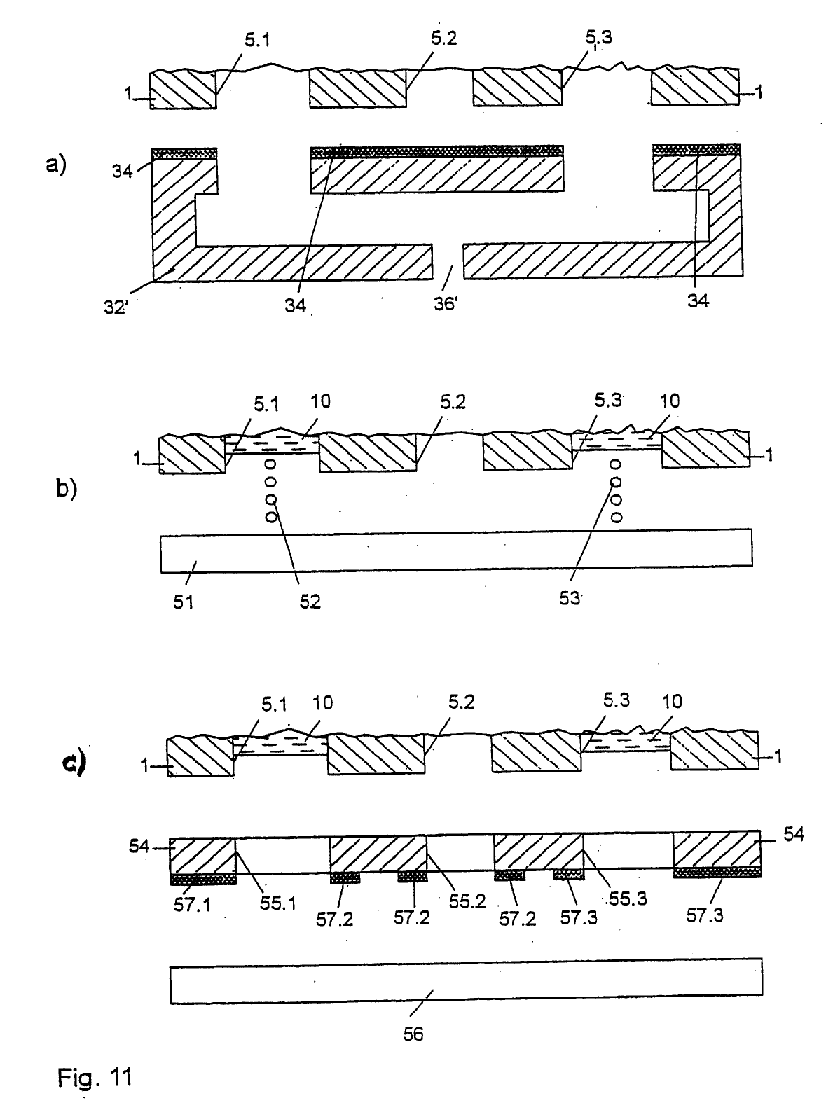

FIG. 11a shows a detail of the control plate 1 according to FIG. 1a with control apertures 5.1 to 5.3. Furthermore, in FIG. 11a is represented a die 32′ which has an aperture 36′ for a control fluid. The die 32′ can now be placed on the control plate 1, and terminates interlocking with the control plate 1, sealing by means of a seal 34. Through the shaping of the die 32′, control apertures 5.1 and 5.3 are left open, whilst control aperture 5.1 is covered. If a control fluid is now introduced at excess pressure through aperture 36′ into the die 32′, through control aperture 5.1 and 5.3 a control fluid domain is formed in the reaction chamber 7, whilst above control aperture 5.1 no control fluid domain is formed.

FIG. 11b shows the introduction of blocking fluid 10 into control apertures 5.1 and 5.3. This comes about here by means of a micro-drop/inkjet device 51, which introduces the blocking fluid 10 in droplet form 52 or 53 into the control aperture 5.1 and 5.3. No blocking fluid is introduced into control aperture 5.2 so that when a control fluid is applied at excess pressure, a control fluid domain will then form above aperture 5.2 in the reaction chamber.

By means of the micro-drop/inkjet method, consequently, for each individual reaction step any desired distribution of blocked and non-blocked control apertures can be produced.

FIG. 11c shows a further possible way of introducing blocking fluid 10 into control apertures 5.1, 5.2 or 5.3. For this purpose, there is arranged on the outer side of the control plate 1 an electrically addressable screen 54, which has screen apertures 55.1, 55.2 or 55.3, which are associated with the control apertures 5.1, 5.2 or 5.3 of the control plate 1. On the outer side of the electrically addressable screen 54 are arranged electrical contacts 57.1 to 57.3, to which an electrical voltage can be applied. At a suitable spacing from the electrically addressable screen 54 is arranged an electrospray source 56, via which electrically charged droplets (not shown here) having a diameter in the sub-micrometer range are deflected onto the electrically addressable screen 54. If now, for example, no electrical voltage is applied to electrical contacts 57.1 and 57.2 around screen aperture 55.1, the droplets fly through the screen apertures 55.1. If an electrical voltage is applied, however, the droplets are deflected according to polarity onto the electrode in 57.1 or 57.2 itself or respectively repelled by same.

In the example of FIG. 11c, by appropriate addressing (application of voltages) of the electrodes, only control aperture 5.1 and 5.3 are filled with a blocking fluid 10.

A further variant for the introduction of the blocking fluid 10 into control apertures 5 consists in the blocking fluid being introduced into the control aperture via a screen printing method (not illustrated here).

FIG. 12 shows a further device according to the invention corresponding to FIG. 5 and in addition the illustration of an optical analysis method. In the device in FIG. 12, fluorophores which can be optically detected are bound at the phase boundary from the analysis interface 3 to the reaction chamber 7. Reference is made to the publication DE 196 28 002 C1 for an analysis method of this type using fluorophores.

Outside the device according to the invention on the side of the analysis interface 3 is disposed a light source 60 which can radiate fluorescence-exciting light 61 onto the analysis interface 3. This takes place at an angle to the normal on the analysis interface 3 which has a value of α. Furthermore, an optical detector 64 is disposed in such a manner that fluorescent light radiated perpendicular to the analysis interface 3 is detected, whilst scattered and reflected light components 62 are not detected by the optical detector 64.

The measurement of the fluorescent light 63 then makes it possible, for example, to detect a fluorophore supplied via the process fluid 4 at the boundary surface between the analysis interface 3 and the reaction 7, with the aid of the optical detector 64.

An alternative optical measuring method which is not shown here consists in scanning the analysis interface 3 by means of laser scanners and scattered light or reflected light components or even fluorescent light components being detected by means of a detector.

FIG. 13a shows a further example of a device according to the invention in which the reaction chamber 7 is enclosed between a first gas-permeable membrane 2 and a second gas-permeable membrane 30. On the first gas-permeable membrane 2 is disposed a control plate 1 corresponding to FIG. 1, which has control apertures 5 to introduce control fluid domains 6 into the reaction chamber 7. On the side of the second gas-permeable membrane 30 associated with the control apertures 5, strip-shaped substrate elements 70, formed from polycarbonate for example, are disposed on the second gas-permeable membrane 30. Syntheses or other chemical reactions can take place on these strip-shaped substrates 70.

FIG. 13a here shows that through the formation of a control fluid domain 6 above a control aperture 5 a strip-shaped substrate 70 is taken out of the reaction chamber, so that a reaction between the process fluid 4 and the strip-shaped substrate 70 is prevented there. In the case of those control apertures 5 which are blocked by a blocking fluid 10 so that no control fluid domain 6 can form, the process fluid 4 is in contact with the substrate 7, such that the desired reaction can take place there.

FIG. 13b shows the strip-shaped substrate 70 in a plan view. It can be recognised that this strip-shaped substrate 70 comprises individual strips 73 which are interconnected transversely via a substrate connecting element 71.

FIG. 13c shows an alternative arrangement of the substrate in which the strip-shaped substrate 70′ is bound in by the second gas-permeable membrane 30′ and the individual strips 70 are connected to one another by the gas-permeable membrane 30′.

The device shown in FIG. 13 makes it possible for example to radiate fluorescence-exciting light into an end surface 72 (see FIG. 13b) of the substrate connecting element 71. If in the course of the analysis reaction fluorophores were bound at the phase boundary between the strip-shaped substrate 70 and the process fluid 4, the substrate connecting element 71 and the strip-shaped substrate 70 guide the fluorescence-exciting light to the fluorophores. The fluorescent light emitted by the fluorophores at the boundary surface of the strip-shaped substrate 70 can be detected with an optical detector 64, as represented in FIG. 13a. This optical detector 64 here extends over the entire reaction chamber. It is however also possible for the optical detector to detect fluorescent light in a spatially dispersed manner, such that for each individual strip-shaped substrate 70 the fluorescence can be separately detected and evaluated.

FIG. 14 shows a device according to the invention which can be actively filled with process fluid by means of a pumping process.

In FIG. 14a is shown a device which corresponds to FIG. 1a. Here two control apertures 5.1 and 5.2 are formed in the control plate.

FIGS. 14b to 14d show the design of the reaction chamber 7 (the channel support) in plan view at different points of time in the pumping process.

The channel support here has the function of a spacer between the gas-permeable membrane 2 and the analysis interface 3.

FIG. 14b shows the two reaction chambers which lie above the respective control apertures 5.1 and 5.2 and which are connected to one another via a connection. The first reaction chamber has a supply line which is provided with a valve V₁ whilst the second reaction chamber has an outflow which is provided with a valve V₂.

At the beginning of the pumping process, valve V₁ and valve V₂ are opened so that process fluid 4.1 can fill the first reaction chamber.

According to FIG. 14c, valve V₁ is then closed and a control fluid domain is pressed into the first reaction chamber, such that the process fluid is forced into the second reaction chamber 4.2.

According to FIG. 14d, there is then produced above the second control aperture 5.2 a control fluid domain which expels the process fluid from the second reaction chamber 4.2 through valve V₂. Altogether, therefore, a pumping action is achieved by the component according to the invention.

Alternatively, although not illustrated here, a pumping action can also be achieved by the control fluid being sucked out of the reaction chamber through one or more control apertures with the aid of negative pressure. Thus, e.g. when valve V₁ is open, more process fluid flows into the device which can then, as described above, be conveyed out through valve V₂.

FIG. 15 shows a further arrangement which corresponds to that in FIG. 14, but in which in addition to a pumping action, a mixing process can also be carried out.

FIG. 15b here shows that the first reaction chamber 49 has two supply lines 45, 46 which are each connected to a valve V₁ or V₂ respectively, with two different process fluids A₁ or A₂. The first reaction chamber 49 here has spacers 15# which are disposed at regular intervals and which also serve as mixing elements to swirl the process fluids introduced into the first reaction chamber 49.

The process fluids A₁ and A₂ are now mixed by being introduced via valves V₁ and V₂ into the first reaction chamber 49 via the control aperture 5.1. To this end, valves V₁ to V₂ are opened.

As these two process fluids A₁ and A₂ flow in, mixing now takes place at the spacers 15#. Then valves V₁ and V₂ are closed and a blocking fluid is introduced into control aperture 5.2.

By means of excess pressure, a control fluid domain is now produced by control aperture 5.1 in the first reaction chamber 49 and presses the mixed process fluids out of the first reaction chamber 49 into the second reaction chamber 50 above the second control aperture 5.2.

Now the blocking fluid is removed from the second control aperture 5.2, for example by evaporation, and a control fluid domain is also produced in the second reaction chamber 50 via the second control aperture 5.2 and transports the mixed process fluids out of the arrangement according to the invention via the open valve V₃.

FIG. 16 describes a device according to the invention, in which a functional layer, for example an immobilised enzyme, which catalyses a substance transformation in the reaction chamber 7 in the presence of a process fluid, is introduced at selected sites.

FIG. 16a here shows a device corresponding to FIG. 1a but without illustrating a process fluid. The gas-permeable membrane 2 of FIG. 1a is here replaced by a membrane support layer 74, for example formed from a net, fabric or porous material.

FIG. 16b shows the application of a functional material 38 to the membrane support layer 74 in the region of the control aperture 5.1. The functional material can here be, for example, an enzyme immobilised on the membrane support layer 74.

FIG. 16c then shows how gas-permeable membrane 39.1 and 39.2, formed from silicon for example, continue to be applied from the liquid phase with subsequent evaporation of the solvent in the regions of the control apertures 5.1 and 5.2 on the side of the membrane support layer 70 remote from the reaction chamber 7.

In the case of a component such as in FIG. 16, the introduction of control fluid domains into the reaction chamber in the region of control aperture 5.1 occurs in the same manner as in the preceding examples. However apertures can also be used which have no control function. This is achieved for example in that 39.2 represents a sealing layer, so that the passage of gas from the outer side of the control plate 1 to the reaction chamber 7 is no longer possible.

FIG. 17a shows a further possible way of generating control fluid domains. In contrast to the previously described arrangement, however, the control fluid is generated electrolytically in-situ inside the reaction chamber 7 from the process fluid 4. To this end two electrodes 40 and 41 are disposed as anodes or cathodes on the control plate 1. By applying an electrical voltage of for example more than 1 volt between the anode 40 and the cathode 41, hydrogen and oxygen gas 42 or 43 are produced in an electrolytic manner from the aqueous process fluid 4. These gases act as the control fluid and form control fluid domains above the electrodes 40 and 41. Altogether, through a device of this type it is possible to delimit the reaction chamber in a locally selective manner and thus keep the process fluid away from, or displace it from, specific regions of the analysis interface 3 or of the control plate.

FIG. 17b now shows a control fluid domain which is produced by joining together two control fluid domains above the electrodes 40 and 41. The size of the control fluid domain 44 consequently depends merely on the duration of the electrochemical decomposition of the process fluid 4, i.e. on the duration of the voltage applied to the electrodes 40 and 41 or respectively also on the level of the voltage applied.

FIG. 17c shows a variant of the device according to the invention via which a delimitation of the control fluid domains is made possible during the electrolytic generation of the control fluid. To this end, the analysis interface is replaced by a second control plate 29′ and a second gas-permeable membrane 30′, which is disposed between the second control plate 29′ and the reaction chamber 7. Opposite the two electrodes 40 and 41 there is arranged in the second control plate an aperture which makes possible free access from outside to the second gas-permeable membrane 30′.

By applying negative pressure to this aperture in the second control plate 29′, the control fluid domains 42 and 43 can be removed. Moreover, by suitable adjustment of the negative pressure, it is possible to limit the expansion of the control fluid domains. The size of the control fluid domains is produced namely inter alia from the relationship between the excess pressure in control fluid domain 42 or 43 and the negative pressure in the aperture in the second control plate 29′.

In further embodiments, not shown here in the figures, instead of optical analysis, an electrochemical detection method can also be used. For this, in known manner, electrodes e.g. a working electrode and a counter-electrode can be applied at the phase boundary between the process fluid 4 and a substrate 3 (see also FIG. 12). Likewise, electrodes can be disposed on the gas-permeable membrane 30′.

As an example of application of the device according to the invention, the production of an array of DNA with different nucleotide sequences is described below.

To this end, in a first step a process fluid is made to flow with a first nucleotide in a spatially selective manner over the array elements of the substrate (of the device) which are not blocked by control fluid domains. The array elements can here be disposed for example as shown in FIG. 4 or FIG. 8.

The nucleotide washed in bears a reactive group which is protected by a protective group and is coupled to the substrate surface, for example at the boundary surface between the reaction chamber 7 and the analysis interface 3 in FIG. 1a, for example in a covalent manner.

Then the first step is repeated with a process fluid which contains a second nucleotide, or with a succession of process fluids which contain second or additional nucleotides. By generating control fluid domains at the individual control apertures it is possible to let the respective process fluid, in a spatially selective manner, reach specific sites inside the reaction chamber. Then the substrate surface is brought into contact, over its entire area or at selected sites, with a reagent to remove the protective groups from the previously coupled nucleotides. Thus it is now only possible at the sites at which the protective group has been removed to couple a further nucleotide to the membrane support layer 70. The above described steps are now carried out with a sequence of process fluids with different nucleotides until each individual point in the array with n lines and m columns has oligonucleotides which have a desired specific nucleotide sequence for each individual point.

Thus the production of oligonucleotide arrays is consequently possible in the simplest manner.

Synthesis can, however, also take place on other substrates such as particles, fabrics, mats, gas-permeable membranes, membrane support layers, electrodes or the like (compare e.g. FIGS. 2b, 2c, 16, 17).

Furthermore the devices and methods according to the invention can be used for epitope analysis/for antibody binding tests. To this end, in an appropriate manner specific antibodies are coupled in a spatially specific manner to individual points of the substrate of the device, as in the above example.

Further possible uses for the devices and methods according to the invention arise in the field of bioassays, e.g. immunoassays, in which here carrying out many assays in a single device according to the invention is now made possible. Here the surface elements of the substrate defined by the control device are functionalised and bear for example biocomponents such as antibodies, antigens, linker molecules and the like.

It is thus possible, to perform a plurality of assays in succession and/or in parallel, it being possible via the control device for the individual surface elements to be addressed individually even during analysis. To this end, the various samples are merely supplied to selected array elements of the substrate which are not blocked by control fluid domains.

The flow-through device according to the invention can be advantageously improved by being temperature-controlled, for example with the aid of a system unit. To this end, various temperature-control methods are available, such as for example making contact with a heating block, irradiation with infrared light or making a temperature-controlled fluid flow around the device. Chemical or biochemical reactions can take place not only on substrates but also in the volume of chambers.

1. Device for performing syntheses, analyses or transport processes with a process fluid having a reaction chamber for accommodating the process fluid which is delimited on two of its opposite sides by a first and a second flat side wall, and having a feed aperture in the reaction chamber to feed the process fluid into the reaction chamber,
wherein
the first and/or the second side wall has at least one control device to introduce a control fluid into the reaction chamber in the region of the control device, and the control device is configured as a control aperture in the form of an opening in the side wall, and the control aperture is closed with a membrane which is permeable by the control fluid, but not by the process fluid.

2. Device according to the claim 1, characterised in that the control apertures have conically configured side walls.

3. Device according to the preceding claim, characterised in that the control aperture tapers in the direction of the reaction chamber.

4. Device according to one of the preceding claims, characterised in that the first and/or the second side wall has at least one suction device for applying a negative pressure to the reaction chamber in the region of the suction device.

5. Device according to the preceding claim, characterised in that the suction device is configured as a suction port in the form of an opening in the side wall.

6. Device according to the preceding claim, characterised in that the suction ports have conically configured side walls.

7. Device according to the preceding claim, characterised in that the suction ports taper in the direction of the reaction chamber.

8. Device according to one of the three preceding claims, characterised in that the suction port is closed with a membrane which is permeable by the control fluid but not by the process fluid.

9. Device according to one of claims 4 to 8, characterised in that the suction device is disposed laterally adjacent to the control device.

10. Device according to one of the preceding claims, characterised in that the control device for introducing the control fluid is configured as a die in the form of a mask for the selection of defined control apertures.

11. Device according to the preceding claim, characterised in that the suction device is disposed completely surrounding the control device in the surface plane of the reaction chamber.

12. Device according to one of the preceding claims, characterised by a blocking device for blocking the control device in such a way that no control fluid can be introduced into the reaction chamber by the control device.

13. Device according to the preceding claim, characterised in that the blocking device has a device for introducing a blocking fluid into the control aperture.

14. Device according to the preceding claim, characterised in that the device for introducing a blocking fluid is configured as a die which can be applied in the form of a mask to the control device.

15. Device according to claim 13, characterised in that the device for introducing a blocking fluid has an electrospray source for the blocking fluid and a mask disposed between the electrospray source and the control device.

16. Device according to claim 13, characterised in that the device for introducing a blocking fluid has a dispensing device or a device for printing the blocking fluid onto the control device.

17. Device according to the preceding claim, characterised in that the device for printing is a micro-drop printing device, an inkjet printing device or a screen printing device.

18. Device according to one of the preceding claims, characterized in that one side wall is configured as the analysis interface.

19. Device according to the preceding claim, characterised in that the analysis interface is light-permeable at least at pre-determined locations.

20. Device according to one of the two preceding claims, characterised in that the analysis interface is filled with analysis reagents at least at pre-determined locations on the side facing the reaction chamber.

21. Device according to one of the preceding claims, characterised in that one side wall is configured as the reaction interface.

22. Device according to the preceding claim, characterised in that the reaction interface is light-permeable at least at pre-determined locations.

23. Device according to one of the two preceding claims, characterised in that the reaction interface, at least at pre-determined locations on the side facing the reaction chamber, is covered with a substrate as the reagent or a substrate is inserted into the reaction interface at least at pre-determined locations.

24. Device according to one of the preceding claims, characterised in that between the two side walls extend webs which divide the reaction chamber into individual reaction chambers which are connected to one another and/or separated from one another.

25. Device according to one of the preceding claims, characterised in that the reaction chamber is divided into at least two interconnected reaction compartments, the first compartment being connected to at least one process media inflow and the second compartment to at least one process media outflow and each of the two compartments being provided with a control device.

26. Device according to the preceding claim, characterised in that each of the at least one process media inflows and/or each of the at least one process media outflows is connected to a valve.

27. Device according to one of the preceding claims, characterised in that the side walls consist at least partially of plastics material, glass, ceramics or the like.

28. Device according to one of the preceding claims, characterised in that the side walls have at least in parts a planar, porous or structured surface.

29. Device according to one of the preceding claims, characterised by a device for controlling the temperature of the reaction chamber and of the process fluid.

30. Device according to the preceding claim, characterised in that the device for controlling the temperature of the reaction chamber has a heating block, an infrared light source and/or a device for making a temperature-controlled fluid flow around the reaction chamber.

31. Device according to one of the preceding claims, characterised in that the membrane consists at least partially of silicon, Teflon, or the like.

32. Device according to one of claims 18 to 31, characterised by at least one light source for radiating light onto the analysis interface and at least one detector for detecting the light reflected, scattered, fluoresced or transmitted by the device.

33. Device according to one of claims 18 to 32, characterised in that the analysis interface consists at least partially of glass, polycarbonate, polyvinyl chloride (PVC), polypropylene (PP), polyurethane (PU), polyester or the like.

34. Device according to one of the preceding claims, characterised in that the side wall consists at least partially of polymers, synthetic resin, polycarbonate, glass, ceramics or the like.

35. Device according to one of the preceding claims, characterised in that the control device has a thickness of 1 μm to 1 mm, the side walls, the reaction face or respectively the analysis interface have a thickness of several μm to several mm, the gas-permeable membrane has a thickness of several 100 nm to several 100 μm and/or the reaction chamber has a height of several 10 μm to 10 mm.

36. Device according to one of the preceding claims, characterised in that the control aperture has a diameter of between 1 μm and 10 mm.

37. Method for performing syntheses, analyses or transport processes, with a device according to at least one of claims 1 to 36, with a process fluid by the process fluid being introduced into THE reaction chamber and there an analysis or a synthesis is performed with the process fluid or the process fluid is transported,
wherein
into at least one predetermined compartment of the reaction chamber the control fluid is introduced in such a way that the process fluid is excluded from this compartment.

38. Method according to the preceding claim, characterised in that the compartment is blocked by the control fluid or the process fluid is displaced from this compartment by the control fluid.

39. Method according to one of the preceding claims, characterized in that the introduction of control fluid into the reaction chamber via a control aperture is blocked by a blocking fluid being introduced into the control aperture.

40. Method according to one of the preceding claims, characterised in that the blocking fluid is introduced into the control aperture by electrospraying, dispensing methods, or printing methods such as micro-drop printing, inkjet printing or screen printing.

41. Method according to one of the two preceding claims, characterized in that a readily volatile medium is used as the blocking fluid.

42. Method according to one of the three preceding claims, characterized in that a liquid medium is used as the blocking fluid.

43. Method according to claim 42, characterized in that the blocking fluid is produced by blowing away from the control interface, e.g. with the aid of a gas flow.

44. Method according to one of the preceding claims, characterised in that the blocking fluid is sucked away by additional channels integrated into the control interface.

45. Method according to one of the four preceding claims, characterised in that water, alcohol, THF or the like is used as the blocking fluid.

46. Method according to claim 37, characterised in that the control fluid is introduced into the reaction chamber at excess pressure.

47. Method according to one of the preceding claims, characterised in that the control fluid is removed from the reaction chamber by the application of negative pressure.

48. Method according to one of the preceding claims, characterised in that a gas is used as the control fluid.

49. Method according to one of the preceding claims, characterised in that a noble gas is used as the control fluid.

50. Method according to one of the preceding claims, characterised in that argon or nitrogen is used as the control fluid.

51. Use of a device according to one of claims 1 to 36 for the transport of fluids, for performing chemical and biochemical reactions for syntheses and analyses, in-situ syntheses, synthesis of detector materials and/or analytes and possibly immediately following analysis in the same device, to generate arrays of various detector materials, as a flow-through synthesis device or as a flow-through analysis device.

52. Use according to the preceding claim for the synthesis of DNA, RNA, oligonucleotides, for epitope analysis, for antibody binding tests, for bioassays such as immunoassays for example.
